(19) United States
(12) Patent Application Publication
(10) Pub. No.: US 2007/0016064 A1
(43) Pub. Date: Jan. 18, 2007

(54) Convex ultrasonic probe and ultrasonic diagnostic apparatus

(76) Inventors: Yohachi Yamashita, Yokohama-shi (JP); Yasuharu Hosono, Yokohama-shi (JP)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(21) Appl. No.: 11/477,470

(22) Filed: Jun. 30, 2006

(30) Foreign Application Priority Data
Jul. 1, 2005 (JP) 2005-193985

(51) Int. Cl. A61B 8/14

(52) U.S. Cl. 600/459

(57) ABSTRACT

A convex ultrasonic probe includes a backing member including a support which has a convexly curved surface and a thermal conductivity of 70 W/m·K or more, and an acoustic absorbent layer having a uniform overall thickness which is fixed on the convexly curved surface of the support, a plurality of channels arranged with spaces on the backing member and each having a piezoelectric element and an acoustic matching layer formed on the piezoelectric element, and an acoustic lens formed on the acoustic matching layer of each of the channels. A relation in which t1/t2=6 to 20 is satisfied, where t1 is the thickness of the acoustic absorbent layer and t2 is the thickness of the piezoelectric element.

### CROSS-REFERENCE TO RELATED APPLICATIONS

This application is based upon and claims the benefit of priority from prior Japanese Patent Application No. 2005-193985, filed Jul. 1, 2005, the entire contents of which are incorporated herein by reference.

### BACKGROUND OF THE INVENTION

1. Field of the Invention

The present invention relates to a convex ultrasonic probe serving to transmit-receive an ultrasonic signal to and from, for example, an object, and an ultrasonic diagnostic apparatus comprising the ultrasonic probe.

2. Description of the Related Art

A medical ultrasonic diagnostic apparatus or ultrasonic image inspecting apparatus transmits an ultrasonic signal to an object and receives an echo signal from within the object so as to form an image of the inside of the object. An array type ultrasonic probe capable of transmitting and receiving an ultrasonic signal is used mainly in these ultrasonic diagnostic and ultrasonic imaging apparatuses.

The array-type ultrasonic probe comprises a backing member, a plurality of channels adhesively bonded onto the backing member and arrayed with spaces, and an acoustic lens adhesively bonded onto the channels. Each of the plurality of channels formed on the backing member comprises a piezoelectric element in which electrodes are applied to both surfaces of a piezoelectric member made of, for example, a lead-zirconium-titanate (PZT)-based piezoelectric ceramic material, and an acoustic matching layer formed on the piezoelectric element. It should be noted that trenches are formed in the backing member to correspond to the spaces of the channels. In performing a medical diagnosis by using the ultrasonic probe, the piezoelectric element is driven under the state that the ultrasonic probe on the side of the acoustic lens contacts against an object so as to transmit an ultrasonic signal from the front surface of the piezoelectric element into the object. The ultrasonic signal is converged at a prescribed position within the object by the electronic focus function produced in accordance with the drive timing of the piezoelectric element and by the focus function produced by the acoustic lens. In this case, it is possible to transmit the ultrasonic signal within a prescribed area within the object by controlling the drive timing of the piezoelectric element, and the echo signal is received from the object and processed in the ultrasonic probe so as to obtain an ultrasonic image (tomographic image) within the prescribed range noted above. The ultrasonic signal is also released to the rear surface by the driving of the piezoelectric element. Therefore, a backing member is arranged on the rear surface of the piezoelectric element absorb (attenuate) the ultrasonic signal transmitted to the rear surface, thereby avoiding a detrimental effect that the normal ultrasonic signal is transmitted into the object together with the ultrasonic signal (echo signal) reflected from the back surface.

Meanwhile, the probes of the ultrasonic diagnostic apparatuses are roughly classified into two types. One is a high-frequency probe or ultrasonic probe for circulatory organs in which a plurality of channels is arranged on a flat-plate-like backing member. The other is an abdominal convex ultrasonic probe in which a plurality of channels is arranged on a backing member having a convex curvature.

JP-A 57-181299(KOKAI) has disclosed a convex ultrasonic probe and a method of manufacturing a convex ultrasonic probe. That is, the convex ultrasonic probe comprises a backing member made of thermoplastic material such as a polyvinyl chloride contained in tungsten powder as filler, the a piezoelectric member affixed to the backing member, and an acoustic matching layer adhesively bonded onto the piezoelectric element. This stack formed with backing member, the piezoelectric member and the acoustic matching layer is cut in an arrayed manner from the acoustic matching layer side to backing member by a dicer to separate a plurality of strips.

When such an ultrasonic probe is driven, ultrasonic energy emitted from the piezoelectric element of the plurality of channels to the backing member side is absorbed and attenuated by the backing member. At this moment, part of the absorbed ultrasonic energy is converted into heat. For example, in the ultrasonic probe for circulatory organs, if the backing member on which the piezoelectric element of the plurality of channels is fixed is flat, ultrasonic waves from the piezoelectric element to the backing member are reflected at the bottom of the backing member in all the channels and then return through an incidence path. That is, the ultrasonic energy is dispersed without concentrating on a particular channel on the backing member. As a result, there is no overheating of the channel located in the center of the backing member alone among the plurality of channels.

However, in the convex ultrasonic probe, ultrasonic waves emitted from the plurality of channels to the backing member side are reflected at the bottom of the backing member and then return to the center thereof in a concentrated manner. Thus, the temperature intensively increases in the channel located in the center of the backing member. Consequently, sensitivity variation and multiple-reflection are caused in the ultrasonic probe. In extreme cases, the object might be thermally affected by the heat generation in the acoustic lens on the surface of the probe.

### BRIEF SUMMARY OF THE INVENTION

According to a first aspect of the present invention, there is provided a convex ultrasonic probe comprising:

a backing member including a support which has a convexly curved surface and a thermal conductivity of 70 W/m·K or more, and an acoustic absorbent layer having a uniform overall thickness which is fixed on the convexly curved surface of the support;

a plurality of channels arranged on the acoustic absorbent layer of the backing member with spaces and each having a piezoelectric element and an acoustic matching layer formed on the piezoelectric element; and

an acoustic lens formed on the acoustic matching layer of each of the channels,

wherein a relation in which t1/t2 is 6 to 20 is satisfied, where t1 is the thickness of the acoustic absorbent layer and t2 is the thickness of the piezoelectric element.

According to a second aspect of the present invention, there is provided an ultrasonic diagnostic apparatus comprising a convex ultrasonic probe and an ultrasonic probe controller connected to the ultrasonic probe through a cable,

the ultrasonic probe comprising:

a backing member including a support which has a convexly curved surface and a thermal conductivity of 70 W/m·K or more, and an acoustic absorbent layer having a uniform overall thickness which is fixed on the convexly curved surface of the support;

a plurality of channels arranged on the acoustic absorbent layer of the backing member with spaces and each having a piezoelectric element and an acoustic matching layer formed on the piezoelectric element; and

an acoustic lens formed on the acoustic matching layer of each of the channels,

wherein a relation in which t1/t2 is 6 to 20 is satisfied, where t1 is the thickness of the acoustic absorbent layer and t2 is the thickness of the piezoelectric element.

### BRIEF DESCRIPTION OF THE SEVERAL VIEWS OF THE DRAWING

FIG. 1 is a perspective view of a partially cut convex ultrasonic probe according to an embodiment;

FIG. 2 is a perspective view of essential parts of the ultrasonic probe of FIG. 1;

FIG. 3 is a sectional view of the essential parts of the ultrasonic probe of FIG. 1;

FIGS. 4A, 4B, 4C and 4D are perspective views showing steps of manufacturing an acoustic absorbent layer according to the embodiment;

FIG. 5 is a schematic view showing an ultrasonic diagnostic apparatus according to the embodiment;

FIG. 6 is a perspective view showing an acoustic backing member used in Example 1 of the present invention; and

FIG. 7 is a perspective view showing an acoustic backing member used in Comparative Example 1.

### DETAILED DESCRIPTION OF THE INVENTION

A convex ultrasonic probe and an ultrasonic diagnostic apparatus according to an embodiment of the present invention will hereinafter be described in detail in reference to the drawings.

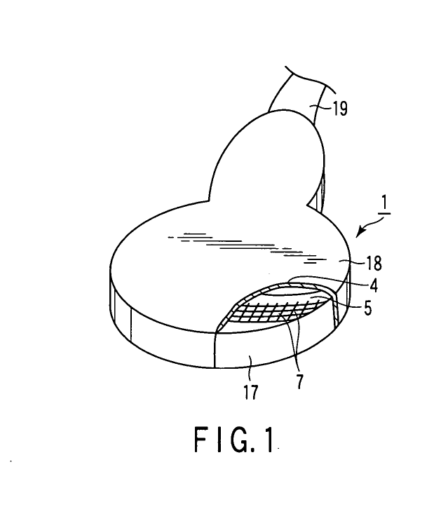

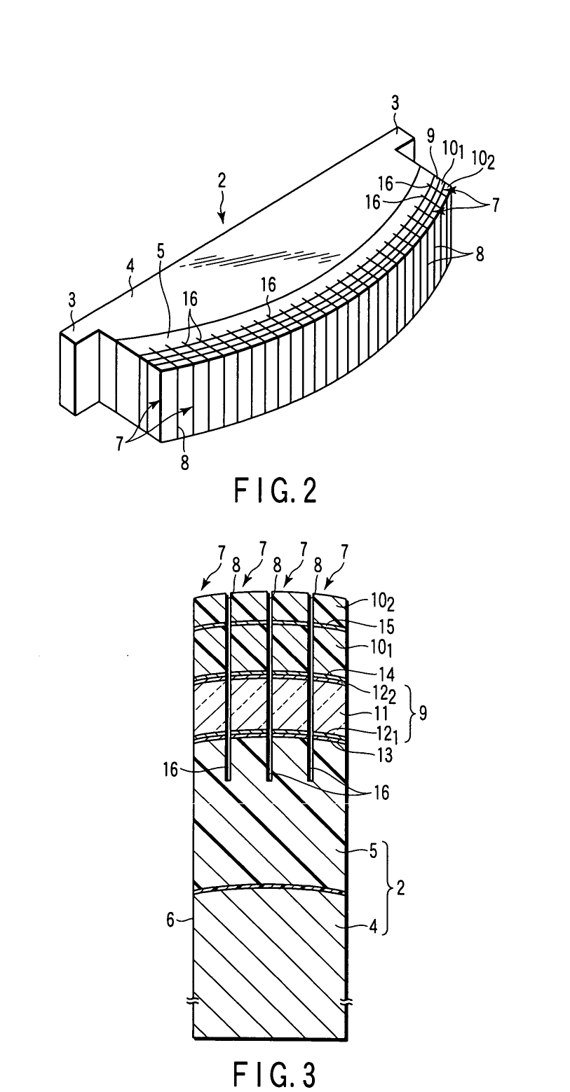

FIG. 1 is a perspective view of a partially cut convex ultrasonic probe according to this embodiment, FIG. 2 is a perspective view of essential parts of the ultrasonic probe of FIG. 1, and FIG. 3 is a sectional view of the essential parts of the ultrasonic probe of FIG. 1.

A convex ultrasonic probe 1 comprises a backing member 2. This backing member 2 includes a support 4 with a convexly curved surface and having rectangular brims 3, 3 at both ends, and an acoustic absorbent layer 5 with a uniform overall thickness fixed on the convexly curved surface of the support 4, as shown in FIGS. 2 and 3. The support 4 has a thermal conductivity of 70 W/m·K or more. The acoustic absorbent layer 5 is adhesively bonded and fixed onto the convexly curved surface of the support 4 by, for example, an epoxy-resin-based adhesive layer 6.

A plurality of channels 7 are arranged on the acoustic absorbent layer 5 of the backing member 2 with spaces 8 at a pitch of, for example, 50 to 200 μm. The channels 7 have a piezoelectric element 9, a first acoustic matching layer 10₁ formed on piezoelectric element 9, and a second acoustic matching layer 10₂ formed on the first acoustic matching layer 10₁. The piezoelectric element 9 comprises a piezoelectric member 11, and first and second electrodes 12₁, 12₂ formed on both surfaces of the piezoelectric member 11, as shown in FIG. 3. The first electrode 12₁ of the piezoelectric element 9 is adhesively bonded and fixed onto the acoustic absorbent layer 5 by, for example, an epoxy-resin-based adhesive layer 13. The first acoustic matching layer 10₁ is adhesively bonded and fixed onto the second electrode 12₂ of the piezoelectric element 9 by, for example, n epoxy-resin-based adhesive layer 14. The second acoustic matching layer 10₂ is adhesively bonded and fixed onto the first acoustic matching layer 10₁ by, for example, an epoxy-resin-based adhesive layer 15. In the acoustic absorbent layer 5 of the backing member 2, trenches 16 are formed in a manner corresponding to the spaces 8 of the plurality of channels 7.

A relation of t1/t2=6 to 20 is satisfied, where t1 is the thickness of the acoustic absorbent layer 5 and t2 is the thickness of the piezoelectric element 9.

An acoustic lens 17 is adhesively bonded and fixed onto the second acoustic matching layer 10₂ of the plurality of channels 7 by, for example, an insulating adhesive layer (not shown) made of an silicone-rubber-based adhesive.

The backing member 2, the plurality of channels 7 and the acoustic lens 17 are housed in a case 18. The case 18 has a built-in signal processing circuit (not shown) including a control circuit which controls the driving timing of the piezoelectric element 9 of the channels 7 and an amplifier circuit which amplifies a signal received in the piezoelectric element 9. A cable 19 connected to the first and second electrodes 12₁, 12₂ extend to the outside of the case 18 on the opposite side of the acoustic lens 17.

In the ultrasonic probe having such a configuration, a voltage is applied across the first and second electrodes 12₁ and 12₂ of the piezoelectric element 9 in the channels 7 so that the piezoelectric member 11 resonates, thereby emitting (transmitting) ultrasonic waves through the acoustic matching layers (the first and second acoustic matching layers 10₁ and 10₂) of the channels 7 and the acoustic lens 17. During reception, the piezoelectric member 11 of the piezoelectric element 9 in the channels 7 is vibrated by the ultrasonic waves received through the acoustic lens 17 and the acoustic matching layers (the first and second acoustic matching layers 10₁ and 10₂) of the channels 7, and this vibration is electrically converted into a signal, thereby obtaining an image.

The support which forms the backing member and has a thermal conductivity of 70 W/m·K or more is made of a metal, for example, an aluminum alloy such as JIS A5052P, 2024, a magnesium alloy such as JIS MT-1, MT-2, a zinc alloy such as JIS ZDC-2, or a copper alloy such as JIS C-1100. The convexly curved surface of this support has a curvature radius of, for example, 20 to 100 mm. The support is not limited to a case of being made of a single material, and may be made of, for example, a composite material of a plastic member and a metal sheet such as a copper sheet. Specifically, the support may be made of a plastic member having a convexly curved surface and a metal sheet formed on the convexly curved surface of the plastic member.

The acoustic absorbent layer forming the backing member is made of, for example, an acoustic absorption compound in which a filler is dispersed in a base material such as an ethylene-vinyl acetate copolymer (EVA), chloroprene rubber, butyl rubber, urethane rubber, silicon rubber, fluorosilicon rubber or fluorinated elastomer. Especially, the base material is preferably the ethylene-vinyl acetate copolymer (EVA) in which the content of vinyl acetate is 20 to 80% by weight.

The filler is contained in the base material in the form of, for example, fiber, woven cloth, powder or flake. This filler contributes to the strength of the acoustic absorbent layer, heat release properties, an improvement in the attenuation rate of the ultrasonic waves, the control of the sound speed, etc.

It is possible to use various fibers as the filler including, for example, at least one selected from the group consisting of carbon fiber, silicon carbide fiber, zinc oxide fiber and alumina fiber. The fiber is not limited to a fiber made of one kind of material, and may be, for example, SiC fiber whose surface is covered with a diamond film by a CVD method or covered with a resin.

The carbon fiber is particularly preferable among the above-mentioned fibers. Various grades of carbon fiber can be used, such as pitch-based carbon fiber and PAN-based carbon fiber. In addition, a carbon nano tube can also be used as the carbon fiber. Especially, pitch-based carbon fiber having a density of 2.1 or more and a thermal conductivity of 100 W/m·K or more is preferable.

The fiber preferably has a diameter of 20 μm or less and a length of five times or more as much as the diameter. The acoustic absorbent layer including a fiber of 20 μm or less in diameter can suppress the reflection from the plurality of channels attached to this acoustic absorbent layer. Moreover, this acoustic absorbent layer is provided with an adequate strength required during dicing. The acoustic absorbent layer including a fiber having a length of five times or more as much as the diameter can increase the heat release properties. For example, when the acoustic absorbent layer is applied to an abdominal probe for 2 to 5 MHz requiring a thickness of 3 mm or more, heat can be effectively released in the acoustic absorbent layer. The upper limit of the length of the fiber is preferably 500 times the diameter thereof.

The powdery filler and the flake-like filler include, for example, at least one inorganic material selected from the group consisting of, zinc oxide, zirconium oxide, aluminum oxide, silicon oxide, titanium oxide, silicon carbide, aluminum nitride, carbon and boron nitride. The powdery filler desirably has an average particle diameter of 30 μm or less, more preferably, 20 μm or less.

The amount of the filler contained in the base material is preferably 20 to 70% by volume based on the total amount of the base material and the filler. If the content of the filler is lower than 20% by volume, it is difficult to effectively improve the mechanical strength, the heat dissipating properties, the attenuation rate and the sound velocity of an acoustic absorbent layer made of an acoustic absorption compound having this amount of the filler. On the other hand, if the filler content exceeds 70% by volume, it is difficult to knead the filler into the base material, and to produce an acoustic absorbent layer having a desired shape from the acoustic absorption compound having this amount of the filler. The more preferable amount of the filler (amount with respect to the total amount of the base material and the filler) is 40 to 60% by volume.

The acoustic absorbent layer allows at least one metal powder selected from the group consisting of tungsten (W), molybdenum (Mo) and silver (Ag) to be further contained therein. The density of the acoustic absorbent layer containing such metal powder becomes higher, and the attenuation rate of the ultrasonic waves can therefore be much higher. It is to be noted that the metal powder is preferably equal to or less than 10% by volume of the total amount of the base material, the filler and the metal powder.

In the acoustic absorbent layer filled with a fiber such as carbon fiber, part of the filled fiber is preferably located in parts between the trenches of the acoustic absorbent layer and in parts between the trenches and a side surface.

Especially, it is preferable that the acoustic absorbent layer is filled with the fiber having a diameter of 20 μm or less and a length of five times or more as much as the diameter in an amount of 20 to 70% by volume, and that 20 to 80% by volume of the fiber in the total filling amount are arranged at an angle of 30° or less to the axis in a thickness direction of the acoustic absorbent layer.

It is preferable that the acoustic absorbent layer has a thickness of 2 to 6 mm, a thermal conductivity at room temperature of 2 W/m·K or more, and an attenuation rate of 3 dB/mmMHz or more.

The density of the acoustic absorbent layer is preferably 2.5 g/cm³ or less. Especially, it is preferable that the acoustic absorbent layer has an acoustic impedance of 2 to 8 MRayls, a thermal conductivity of 2 W/m·K or more, more preferably 5 W/m·K, and a density of 2.5 or less.

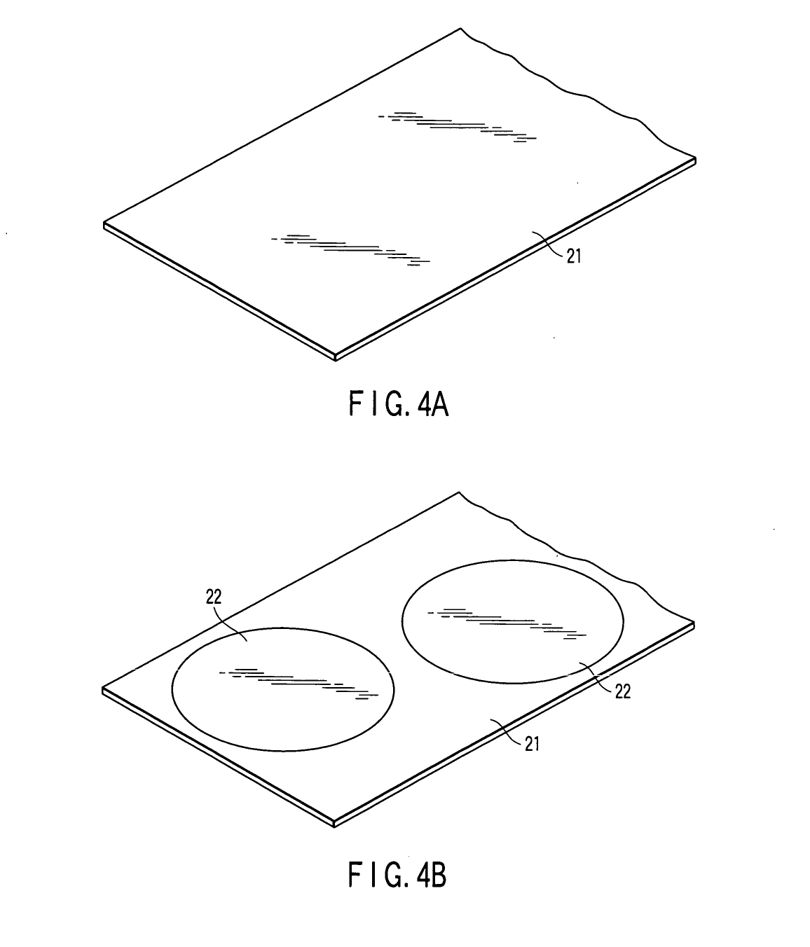

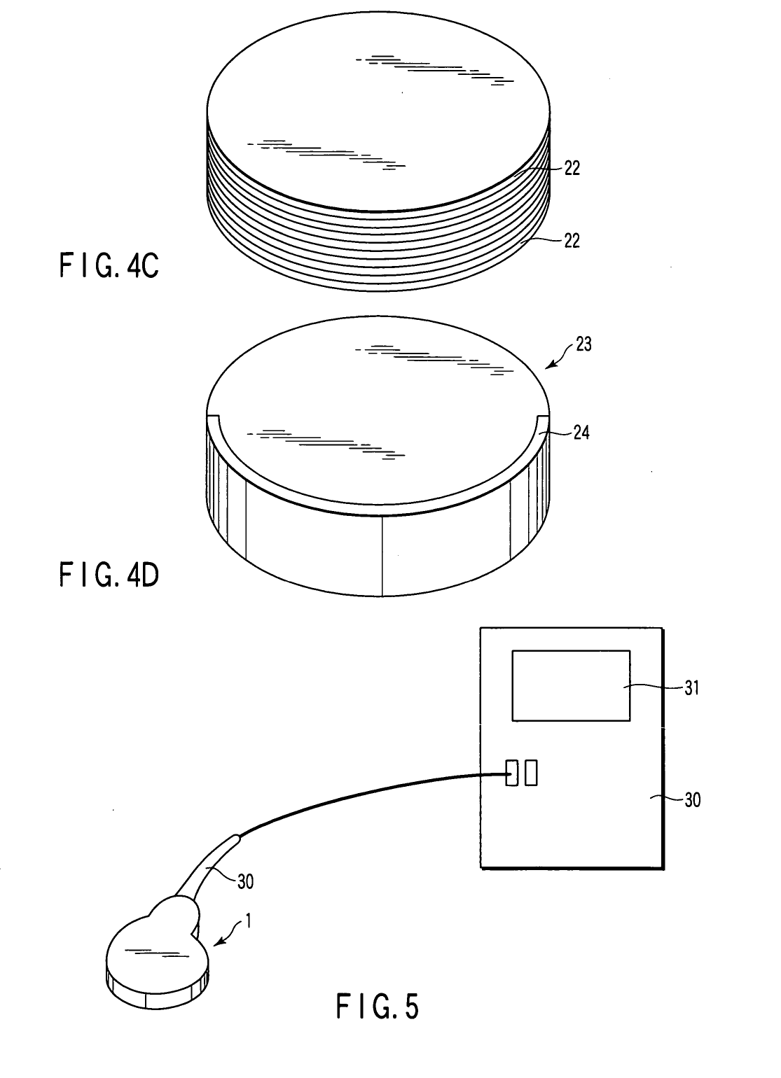

A method of manufacturing such an acoustic absorbent layer (whose base material is EVA) will be described in reference to FIGS. 4A to 4D.

First, EVA in which the content of vinyl acetate is, for example, 20 to 80% by volume is placed between heated rolls and kneaded, and then a filler, a vulcanizing agent, a vulcanization accelerator, etc. are added to EVA, and they are kneaded and formed into a sheet, thereby forming a sheet 21 as shown in FIG. 4A. The thickness of the sheet 21 is preferably 0.5 to 1.0 mm. Subsequently, the sheet 21 is, for example, circularly punched as shown in FIG. 4B to cut out a plurality of circular sheets 22. Then, the plurality of circular sheets 22 cut out is stacked as shown in FIG. 4C to form a stack 23. This stack 23 is heated at, for example, 120 to 180° C. to vulcanize (bridge) the circular sheets 22 so that a circular-disc-shaped block 24 of, for example, 10 to 30 mm in thickness is produced as shown in FIG. 4D, and then this circular-disc-shaped block 24 is cut along its outer peripheral surface vertically to a circular surface thereof, thereby cutting out an acoustic absorption material 25 having a desired R. Subsequently, the acoustic absorption material 25 is cut into desired dimensions, thereby manufacturing an acoustic absorbent layer (not shown).

Especially, in the method described above, if use is made of an acoustic absorption compound which contains EVA as the base material and which contains a fiber (carbon fiber) having a diameter of 20 μm or less and a length of five times or more as much as the diameter in an amount of 20 to 70% by volume, it is possible to obtain an acoustic absorbent layer in which 20 to 80% by volume of the fiber in the total filling amount is arranged at an angle of 30° or less to the axis in the thickness direction.

In the acoustic absorbent layer, if a shield made of a metal such as copper or silver is disposed on the side surface thereof, the provision of further heat release properties is permitted. Moreover, if a ground polar wire or a shield wire of the cable to be connected to a signal electric terminal or a ground electric terminal is brought into contact with the acoustic absorbent layer, the heat release properties of the acoustic absorbent layer can be improved.

In the ultrasonic probe according to the embodiment, a relation of t1/t2=6 to 20 is satisfied, where t1 is the thickness of the acoustic absorbent layer and t2 is the thickness of the piezoelectric element. When t1/t2 is below 6, it becomes difficult to sufficiently attenuate the ultrasonic waves emitted from the piezoelectric element of the plurality of channels to its rear side, the multiple-reflection might be caused. On the other hand, when t1/t2 is over 20, the properties of heat release from the acoustic absorbent layer to the support having a predetermined thermal conductivity deteriorate, so that a temperature increase in the acoustic lens and sensitivity variation among the channels might be greater. t1/t2 is more preferably 8 to 15. The acoustic absorbent layer preferably has a thickness of 2 to 6 mm under the above-mentioned relation of t1/t2.

The piezoelectric member forming the piezoelectric element is made of, for example, PZT-based or relaxer-based piezoelectric ceramics, relaxer-based single crystal or the like, and a composite material of these materials and a resin.

The first and second electrodes are formed in a method of, for example, baking a paste containing gold, silver and nickel powders onto both surfaces of the piezoelectric member, sputtering gold, silver and nickel, or plating with gold, silver and nickel.

The first and second acoustic matching layers are made of, for example, a material based on an epoxy resin. The acoustic matching layer is not limited to multiple layers including two or more layers, and can also be used in the form of one layer.

The acoustic lens is made of, for example, a silicone-rubber-based material.

Next, a method of manufacturing the ultrasonic probe according to the embodiment will be described.

First, the acoustic absorbent layer, the piezoelectric element, the first acoustic matching layer and the second acoustic matching layer are stacked in this order on the support, and, for example, an epoxy-resin-based adhesive is placed between the stacked components. The acoustic absorbent layer can be manufactured in accordance with, for example, the above-mentioned method in FIGS. 4A to 4D. Subsequently, the stack is heated at, for example, 120° C. for about one hour to cure the epoxy-resin-based adhesive, thereby adhesively bonding and fixing, by insulating adhesive layers, the support to the acoustic absorbent layer, the acoustic absorbent layer to the piezoelectric element, the piezoelectric element to the first acoustic matching layer, the first acoustic matching layer to the second acoustic matching layer.

Next, the stack is diced with a diamond saw at a width (pitch) of, for example, 50 to 200 μm from the second acoustic matching layer to the acoustic absorbent layer of the backing member to divide the stack into a plurality of parts in an arrayed manner, thereby forming a plurality of channels having the piezoelectric element, the first and second acoustic matching layers. At this moment, trenches are formed in the acoustic absorbent layer of the backing member in a manner corresponding to the spaces of the plurality of channels. Then, the acoustic lens is adhesively bonded and fixed to the second acoustic matching layer of each of the channels by a silicone-rubber-based adhesive layer, and the backing member comprising the support and the acoustic absorbent layer, the plurality of channels and the acoustic lens are put into the case, thereby manufacturing the ultrasonic probe.

The ultrasonic diagnostic apparatus comprising the ultrasonic probe according to the embodiment of the present invention will be described in reference to FIG. 5.

The medical ultrasonic diagnostic apparatus (or ultrasonic imaging test apparatus) transmits an ultrasonic signal to a object and receives a reflected signal (echo signal) from the object to image the object, and this apparatus comprises the array-type convex ultrasonic probe 1 having an ultrasonic signal transmitting/receiving function. This ultrasonic probe 1 has the structure shown in FIGS. 1 to 3 described above. The ultrasonic probe 1 is connected to an ultrasonic diagnostic apparatus body 30 through the cable 19. The ultrasonic diagnostic apparatus body 30 is provided with a display 31.

The convex ultrasonic probe according to the embodiment described above comprises the backing member having the support with a convexly curved surface and a thermal conductivity of 70 W/m·K or more and the acoustic absorbent layer with a uniform overall thickness disposed on the curved surface of the support, such that the acoustic absorbent layer of the backing member can absorb and attenuate the ultrasonic waves produced by the driving of the piezoelectric element of the plurality of channels and emitted to the rear side of the piezoelectric element. At the same time, heat produced in the piezoelectric element and heat produced due to the attenuation of the ultrasonic waves in the acoustic absorbent layer can be well released to the outside by the support of the backing member having a good thermal conductivity. In such emission of the ultrasonic waves to the rear surface of the channels (piezoelectric element), the thickness of the acoustic absorbent layer is prescribed by t1/t2=6 to 20 (t1: the thickness of the acoustic absorbent layer, t2: the thickness of the piezoelectric element) in relation to the thickness of the piezoelectric element (frequency of the ultrasonic waves), thereby making it possible to effectively attenuate the energy of the ultrasonic waves in the acoustic absorbent layer and well release the produced heat to the outside.

Furthermore, regarding the multiple-reflection of the ultrasonic waves, when the backing member is, as heretofore, made exclusively of a composite in which a filler such as a fiber or powder is contained in a base material such as chloroprene rubber, the ultrasonic waves concentrate on the surface in the center of the backing member due to the reflection, thus raising the temperature of the surface. As in the embodiment, the backing member has a configuration carrying the support with a convexly curved surface and a good thermal conductivity and the acoustic absorbent layer with a uniform overall thickness disposed on the curved surface of the support, whereby the ultrasonic waves traveling from the piezoelectric element to the backing member pass through the acoustic absorbent layer having the equal overall thickness in all the channels, and are reflected by the support having the convexly curved surface, and then return through an incidence path. That is, the reflected ultrasonic waves return to an ultrasonic incidence point in a dispersed manner without concentrating on the center of the acoustic absorbent layer, thereby allowing a uniform heat generation state of the acoustic absorbent layer.

Therefore, it is possible to well attenuate the ultrasonic waves emitted from the piezoelectric element of the plurality of channels to the backing member on the rear side of the piezoelectric element, so that the occurrence of the multiple-reflection can be prevented. As a result, the quality of a cross-sectional image can be improved in the ultrasonic diagnostic apparatus incorporating this ultrasonic probe.

Furthermore, the heat produced in the piezoelectric element and the heat produced due to the attenuation of the ultrasonic waves in the acoustic absorbent layer can be well released to the outside by the support of the backing member having a good thermal conductivity, and the concentration on the center of the acoustic absorbent layer due to the reflection can be avoided to achieve a uniform heat generation state of the acoustic absorbent layer. As a result, the sensitivity variation among the channels can be suppressed. Moreover, an excessive temperature increase in the center of the acoustic lens can be prevented to maintain a low surface temperature of the acoustic lens layer, thereby allowing a good application to the abdominal probe. In addition, the ultrasonic diagnostic apparatus incorporating the ultrasonic probe maintaining a lower surface temperature is capable of raising a transmission voltage, so that a distance in an observable diagnostic region can be extended, and, for example, it becomes possible to observe deep parts of a human body.

Especially, when the acoustic absorbent layer is in such a form that a fiber having a diameter of 20 μm or less and a length five times or more as much as the diameter are filled in an amount of 20 to 70% by volume and that 20 to 80% by volume of the fiber in the total filling amount are arranged at an angle of 30° or less to the axis in the thickness direction, a high attenuation rate of the ultrasonic waves can be realized. That is, if a significant amount of the fiber filled in the base material such as EVA is arranged in the acoustic absorbent layer of the backing member in the thickness direction thereof, that is, arranged in a traveling direction of the ultrasonic waves when the ultrasonic waves generated in the piezoelectric element of the plurality of channels are emitted to the rear backing member, efficient attenuation is surprisingly attained while the ultrasonic waves are transmitted through the fiber, thus allowing the realization of a higher attenuation rate. Especially, if carbon fiber is selected among fibers, a much higher attenuation rate can be realized.

Furthermore, in the acoustic absorbent layer having the configuration described above, the strengths in the thickness and surface directions can be balanced by the arrangement of the filled carbon fiber, such that stress during dicing can be well relieved and cracks can thus be prevented from being caused. As a result, it is possible to more effectively prevent a failure in the channels.

Furthermore, filling the fiber can further improve the heat release properties in the acoustic absorbent layer having the configuration described above. Especially, if the carbon fiber is selected, a further improvement can be significantly attained in the heat release properties.

Still further, in the acoustic absorbent layer in which the arrangement of the fiber such as the carbon fiber is prescribed, if part of the filled fiber is located between the trenches and in parts between the trenches and the side surface, bending can be more effectively prevented in the parts of the backing member between the trenches and between the trenches and the side surface. As a result, it is possible to more effectively prevent a failure in the channels during dicing.

Examples of the present invention will be described below.

### EXAMPLE 1

First, an ethylene-vinyl acetate copolymer (EVA) in which the blend amount of vinyl acetate was 50% by weight was supplied between heated rolls heated to about 70° C., and pre-kneaded for 20 minutes. Subsequently, carbon fiber (filler) having an average diameter of 10 μm and an average length of 20 mm, dioctylsebacate, 6 parts by weight of a vulcanizing agent, 2 parts by weight of zinc stearate (vulcanization accelerator), 4 parts by weight of Carbana Wax and 3 parts by weight of silicon resin were added to 100 parts by weight of pre-kneaded EVA. They were further kneaded and processed for sheet formation for 20 minutes, thereby producing a sheet 400 mm wide and 0.5 mm thick. It should be noted that pitch-based carbon fiber having a thermal conductivity of 500 W/m·K were used as the carbon fiber, and the carbon fiber was blended into the kneaded material in an amount of 50% by volume. Subsequently, circular plates having a diameter of 100 mm were cut out from the sheet. After 40 circular sheets were stacked, the stack was placed in a mold, heated and vulcanized at 180° C. under pressure for 15 minutes, thereby manufacturing a circular-disc-shaped block having a diameter of 100 mm and a thickness of 13 mm. The circular-disc-shaped block was sliced along its outer peripheral surface so that the thickness might be 4 mm when viewed from a direction vertical to a circular surface thereof, thus obtaining a circular arc-shaped slice (acoustic absorbent layer) having a circular arc length of 70 mm, a width of 20 mm and a thickness (t1) of 4 mm. This acoustic absorbent layer had a structure in which 20% by volume of the total filling amount of the carbon fiber was arranged at an angle of 30° or less to the axis in the thickness direction thereof.

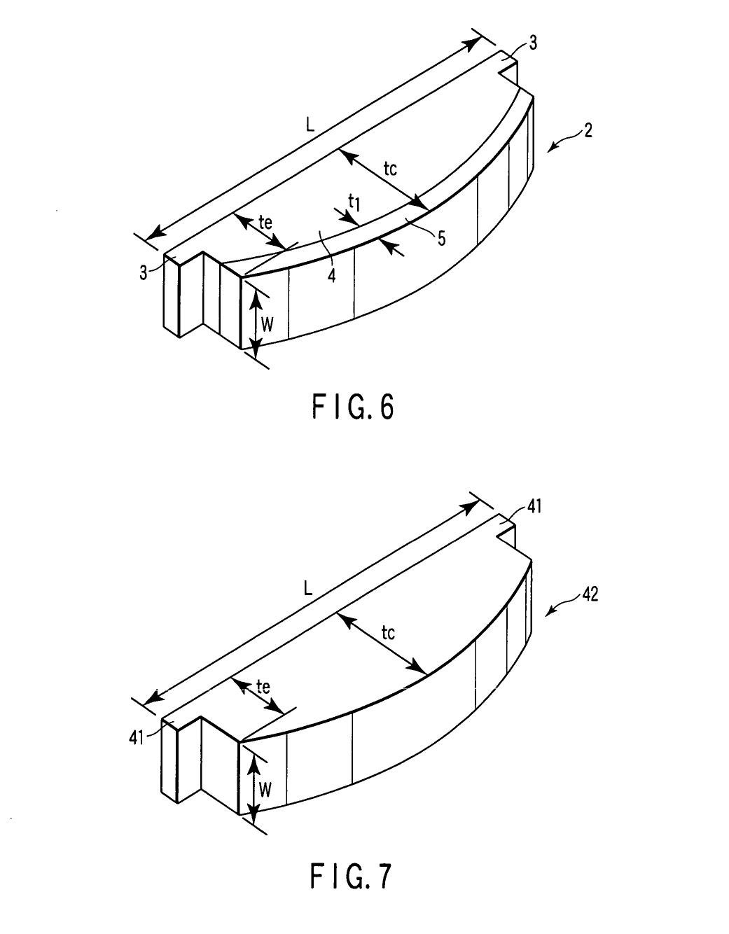

Next, as shown in FIG. 6, a support 4 made of a JIS A5052P aluminum alloy (thermal conductivity: 150 W/m·K) was prepared, and this support 4 had brims 3, 3 of 1 mm in thickness and 4 mm in length at both ends thereof, and had a convexly curved surface (R=44 mm) in the front, and had a total length (L) including the brims 3, 3 of 70 mm and a width (W) of 13 mm. A circular arc-shaped acoustic absorbent layer 5 (thickness (t1): 4 mm) was fixed to the convexly curved surface of the support 4 by an epoxy-resin-based adhesive, thereby manufacturing a backing member 2. It should be noted that this backing member 2 had a thickness (te) of 10.5 mm from the front surface (the surface of the acoustic absorbent layer 5) to the rear surface at ends where the brims 3, 3 extended, and a thickness (tc) of 20.6 mm from the front surface (the surface of the acoustic absorbent layer 5) to the rear surface in the center.

Next, a piezoelectric element, a first acoustic matching layer having an acoustic impedance of 7.5 MRayls in which 40% by volume of alumina is added to an epoxy resin, and a second acoustic matching layer made of an epoxy resin and having an acoustic impedance of 3.5 MRayls were stacked in this order on the front surface of the backing member having the convexly curved surface while the epoxy-resin-based adhesive was placed in between, and then these members were heated and cured at 120° C. for about one hour so that they are adhesively bonded to each other. Subsequently, they were diced from the second acoustic matching layer to the backing member so that the width might be 50 μm and the depth of cuts in the backing member might be 200 μm, thereby forming 2 lines of 200 channels (400 channels in total). Then, an acoustic lens made of silicon rubber and having an acoustic impedance of 1.5 MRayls was fixed onto each of the channels by the epoxy-resin-based adhesive to assemble a simulative test product of an ultrasonic probe. It should be noted that the piezoelectric element used had a structure in which first and second electrodes made of Ni were formed on both surfaces of PZT-based piezoelectric ceramics (piezoelectric member).

### EXAMPLE 2

A backing member similar to that in Example 1 was manufactured except that the thickness of an acoustic absorbent layer was 5 mm. It should be noted that the outside dimensions of this backing member were the same as those in Example 1, and because the acoustic absorbent layer had a large thickness of 5 mm, the thickness of a support was reduced correspondingly. Moreover, a simulative test product of an ultrasonic probe similar to that in Example 1 was assembled by use of this backing member.

### REFERENCE EXAMPLE 1

A backing member similar to that in Example 1 was manufactured except that the thickness of an acoustic absorbent layer was 2 mm. It should be noted that the outside dimensions of this backing member were the same as those in Example 1, and because the acoustic absorbent layer had small thickness of 2 mm, the thickness of a support was increased correspondingly. Moreover, a simulative test product of an ultrasonic probe similar to that in Example 1 was assembled by use of this backing member.

### REFERENCE EXAMPLE 2

A backing member similar to that in Example 1 was manufactured except that the thickness of an acoustic absorbent layer was 9 mm. It should be noted that the outside dimensions of this backing member were the same as those in Example 1, and because the acoustic absorbent layer had a large thickness of 9 mm, the thickness of a support was reduced correspondingly. Moreover, a simulative test product of an ultrasonic probe similar to that in Example 1 was assembled by use of this backing member.

### COMPARATIVE EXAMPLE 1

First, an ethylene-vinyl acetate copolymer (EVA) in which the blend amount of vinyl acetate was 50 percent by weight was supplied between heated rolls heated to about 70° C., and pre-kneaded for 20 minutes. Subsequently, carbon fiber (filler) having an average diameter of 10 μm and an average length of 20 mm, dioctylsebacate, 6 parts by weight of a vulcanizing agent, 2 parts by weight of zinc stearate (vulcanization accelerator), 4 parts by weight of Carbana Wax and 3 parts by weight of silicon resin were added to 100 parts by weight of pre-kneaded EVA, and they were further kneaded. This kneaded material was placed in a mold, heated and vulcanized at 180° C. under pressure for 15 minutes, and then the outer shape thereof was processed, thereby manufacturing a backing member 42 shown in FIG. 7. This backing member 42 had the same outside dimensions as those in Example 1, that is, outside dimensions including a thickness (te) of 10.5 mm from the front surface to the rear surface at ends where brims 41, 41 extended and a thickness (tc) of 20.6 mm from the front surface to the rear surface in the center. It is to be noted that pitch-based carbon fiber having a thermal conductivity of 500 W/m·K were used, and the carbon fiber was blended into the kneaded material in an amount of 50% by volume.

Furthermore, a simulative test product of an ultrasonic probe similar to that in Example 1 was assembled by use of this backing member.

Measurements were made concerning the attenuation rate of the obtained acoustic absorbent layers forming the backing member in Examples 1 and 2, Reference Examples 1 and 2 and Comparative Example 1, and the thermal conductivity of the acoustic absorbent layers forming the backing member. It should be noted that acoustic absorbent layer of Comparative Example 1 was use as the backing member. Moreover, the temperature of the acoustic lens, sensitivity variation among the channels, and multiple-reflection were checked by use of the simulative test products of the ultrasonic probe.

It should be noted that the attenuation rate, the thermal conductivity, the temperature of the acoustic lens, sensitivity variation among the channels, and multiple-reflection were measured in the following manner.

1) Attenuation Rate

The attenuation rate was measured in an underwater method at 25° C. by use of a probe (a measurement frequency of 3.0 MHz) concerning samples of 1.0 mm in thickness cut out from the acoustic absorbent layers forming the backing member in Examples 1 and 2, Reference Examples 1 and 2 and Comparative Example 1.

2) Thermal Conductivity

The thermal conductivity was measured in a laser flash method. The measured sample was 1.0 mm in thickness and 10.0 mm in diameter.

3) Temperature of the Acoustic Lens

The temperature of the acoustic lens was measured in the following manner: a thermocouple was affixed to the surface of the lens which hand been formed into a probe and continuously driven in the air with a transmission voltage of 100 V, and then the surface temperature thereof in 30 minutes was measured. The measurement was made at a room temperature of 20° C.

4) Sensitivity Variation Among the Channels

For the sensitivity variation among the channels of the probe, transmission/receiving sensitivities were measured for each of the channels, and its variation from an average value was expressed by percent.

5) Multiple-Reflection

For measurement of the multiple-reflection, a phantom placed in water was observed by use of the probe, and the presence of the multiple-reflection was checked for in accordance with the image of the phantom.

The results are shown in Table 1 below. It should be noted that Table 1 below includes the material of the support of the backing member, the thickness of the acoustic absorbent layer (t1), the thickness of the piezoelectric element (t2) and t1/t2.

TABLE 1

| | Backing member | | | | | | | | | |
|---|---|---|---|---|---|---|---|---|---|---|
| | | Acoustic absorbent layer | | | Thickness of | | | Acoustic | | |
| | Material | | Thickness: | Thermal | piezoelectric | | Attenuation | lens | Sensitivity | Presence of |
| | of | | t1 | conductivity | element: t2 | | rate | temperature | variation | Multiple- |
| | support | Material | (mm) | (W/m · K) | (mm) | t1/t2 | (dB/mmMHz) | (° C.) | (%) | reflection |
| Example 1 | Al alloy | EVA + 50 | 4 | 15.2 | 0.4 | 10 | 5.20 | 35 | 2.1 | No |
| | | vol % of | | | | | | | | |
| | | carbon | | | | | | | | |
| | | fibers | | | | | | | | |
| Example 2 | Al alloy | EVA + 50 | 5 | 15.2 | 0.4 | 12.5 | 5.20 | 37 | 2.1 | No |
| | | vol % of | | | | | | | | |
| | | carbon | | | | | | | | |
| | | fibers | | | | | | | | |
| Reference | Al alloy | EVA + 50 | 2 | 15.2 | 0.4 | 5 | 5.20 | 37 | 4.9 | Yes |
| Example 1 | | vol % of | | | | | | | | |
| | | carbon | | | | | | | | |
| | | fibers | | | | | | | | |
| Reference | Al alloy | EVA + 50 | 9 | 15.2 | 0.4 | 22.5 | 5.20 | 41 | 14.2 | No |
| Example 2 | | vol % of | | | | | | | | |
| | | carbon | | | | | | | | |
| | | fibers | | | | | | | | |

| | | | | | | | |
|---|---|---|---|---|---|---|---|
| Comparative | EVA + 50 vol % of carbon fiber | 0.4 | — | 5.20 | 42 | 15.6 | No |
| Example 1 | (without support) | | | | | | |
| | Thermal conductivity of 15.2 W/m · K | | | | | | |

The simulative test products of the ultrasonic probe in Examples 1 and 2 comprise the backing member in which the acoustic absorbent layer with a uniform overall thickness containing a predetermined amount of carbon fiber in EVA is fixed to the support having the convexly curved surface and a thermal conductivity of 70 W/m·K or more, and these simulative test products satisfy the relation where the ratio (t1/t2) of the thickness of the acoustic absorbent layer (t1) to the thickness of the piezoelectric element (t2) is 6 to 20. As apparent from Table 1 above, in these simulative test products, a lower temperature can be maintained in the acoustic lens, the sensitivity variation among the channels is small, and the multiple-reflection, which occurs in Example 1, does not occur, as compared with the simulative test product of the ultrasonic probe in Comparative Example 1 comprising the backing member having the same dimensions as those in Examples 1 and 2 in which predetermined amount of carbon fiber is contained in EVA.

On the other hand, in the simulative test product of the ultrasonic probe in Reference Example 1 in which t1/t2 in the acoustic absorbent layer of the backing member is below 6 (t1/t2=5), a lower temperature can be maintained in the acoustic lens, and the sensitivity variation among the channels is slightly small, but the multiple-reflection occurs. The occurrence of the multiple-reflection is due to the insufficient attenuation of the ultrasonic waves because the thickness of the acoustic absorbent layer is smaller than the thickness of the piezoelectric element by relative comparison.

Furthermore, in the simulative test product of the ultrasonic probe in Reference Example 2 in which t1/t2 in the acoustic absorbent layer of the backing member is over 20 (t1/t2=22.5), there is no occurrence of the multiple-reflection, but the temperature of the acoustic lens increases, and the sensitivity variation among the channels thus is greater. The temperature increase in the acoustic lens is due to a low thermal conductivity and the insufficient heat release properties for the piezoelectric element because the thickness of the acoustic absorbent layer is larger than the thickness of the piezoelectric element by relative comparison.

### EXAMPLES 3 TO 7

A backing member similar to that in Example 1 was manufactured except that materials shown in Table 2 below were used for a support of the backing member and an acoustic absorbent layer. Further, simulative test products of an ultrasonic probe similar to that in Example 1 were assembled.

An examination was made by a measuring method similar to that in Example 1 concerning the attenuation rate of the obtained acoustic absorbent layers forming the backing member in Examples 3-7, the thermal conductivity, the temperature of the acoustic lens in accordance with the simulative test products of the ultrasonic probe, sensitivity variation among the channels, and multiple-reflection. The results are shown in Table 2 below. It should be noted that Table 2 below includes the material of the support of the backing member, the thickness of the acoustic absorbent layer (t1), the thickness of the piezoelectric element (t2) and t1/t2.

TABLE 2

| | Backing member | | | | | | | | | |
|---|---|---|---|---|---|---|---|---|---|---|
| | | Acoustic absorbent layer | | | Thickness of | | | Acoustic | | |
| | Material | | Thickness: | Thermal | piezoelectric | | Attenuation | lens | Sensitivity | Presence of |
| | of | | t1 | conductivity | element: t2 | | rate | temperature | variation | Multiple- |
| | support | Material | (mm) | (W/m · K) | (mm) | t1/t2 | (dB/mmMHz) | (° C.) | (%) | reflection |
| Example 3 | Mg alloy | EVA + 50 | 4 | 15.2 | 0.4 | 10 | 5.20 | 35 | 2.1 | No |
| | | vol % of | | | | | | | | |
| | | carbon | | | | | | | | |
| | | fibers | | | | | | | | |
| Example 4 | Zn alloy | EVA + 50 | 4 | 15.2 | 0.4 | 10 | 5.20 | 37 | 2.1 | No |
| | | vol % of | | | | | | | | |
| | | carbon | | | | | | | | |
| | | fibers | | | | | | | | |
| Example 5 | Cu alloy | EVA + 50 | 4 | 15.2 | 0.4 | 10 | 5.20 | 33 | 2.2 | No |
| | | vol % of | | | | | | | | |
| | | carbon | | | | | | | | |
| | | fibers | | | | | | | | |
| Example 6 | Al alloy | EVA + 25 | 5 | 10.2 | 0.4 | 12 | 5.0 | 36 | 1.8 | No |
| | | vol % of | | | | | | | | |
| | | carbon | | | | | | | | |
| | | fibers + | | | | | | | | |
| | | 25 vol % | | | | | | | | |
| | | of ZnO | | | | | | | | |
| | | fibers | | | | | | | | |
| Example 7 | Al alloy | EVA + 40 | 4 | 3.5 | 0.4 | 10 | 4.8 | 38 | 1.5 | No |
| | | vol % of | | | | | | | | |
| | | ZnO | | | | | | | | |
| | | fibers | | | | | | | | |

As apparent from Table 2 above, in the simulative test products of the ultrasonic probe in Examples 3 to 7, the material of the support of the backing member with the convexly curved surface and the material of the acoustic absorbent layer with a uniform overall thickness fixed to the support are changed, and these simulative test products satisfy the relation where the ratio (t1/t2) of the thickness of the acoustic absorbent layer (t1) to the thickness of the piezoelectric element (t2) is 6 to 20. It is thus understood that these simulative test products have good characteristics, as in Example 1, so that a low temperature can be maintained in the acoustic lens, the sensitivity variation among the channels is small, and the multiple-reflection does not occur.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general invention concept as defined by the appended claims and their equivalents.

What is claimed is:

1. A convex ultrasonic probe comprising:
a backing member including a support which has a convexly curved surface and a thermal conductivity of 70 W/m·K or more, and an acoustic absorbent layer having a uniform overall thickness which is fixed on the convexly curved surface of the support;
a plurality of channels arranged on the acoustic absorbent layer of the backing member with spaces and each having a piezoelectric element and an acoustic matching layer formed on the piezoelectric element; and
an acoustic lens formed on the acoustic matching layer of each of the channels,
wherein a relation in which t1/t2 is 6 to 20 is satisfied, where t1 is the thickness of the acoustic absorbent layer and t2 is the thickness of the piezoelectric element.

2. The probe according to claim 1, wherein the support is made of a metal.

3. The probe according to claim 1, wherein the support includes a plastic member having a convexly curved surface and a metal sheet formed on the convexly curved surface of the plastic member and having a uniform thickness.

4. The probe according to claim 1, wherein the convexly curved surface of the support has a curvature radius of 20 to 100 mm.

5. The probe according to claim 1, wherein the acoustic absorbent layer includes an ethylene-vinyl acetate copolymer and at least one filler selected from the group consisting of a fiber and inorganic material powder contained in the ethylene-vinyl acetate copolymer.

6. The probe according to claim 5, wherein an amount of vinyl acetate in the ethylene-vinyl acetate copolymer is 20 to 80% by weight.

7. The probe according to claim 5, wherein the fiber has a diameter of 20 μm or less and a length of five times or more as much as the diameter.

8. The probe according to claim 5, wherein the fiber is at least one selected from the group consisting of carbon fiber, silicon carbide fiber, zinc oxide fiber and alumina fiber.

9. The probe according to claim 5, wherein the fiber is contained in the ethylene-vinyl acetate copolymer in an amount of 20 to 70% by volume based on the total amount of the ethylene-vinyl acetate copolymer and the fiber.

10. The probe according to claim 5, wherein the inorganic material is at least one selected from the group consisting of zinc oxide, zirconium oxide, aluminum oxide, silicon oxide, titanium oxide, silicon carbide, aluminum nitride, carbon and boron nitride.

11. The probe according to claim 1, wherein the acoustic absorbent layer includes a chloroprene-based resin and at least one filler selected from the group consisting of a fiber and inorganic material powder contained in the chloroprene-based resin.

12. The probe according to claim 11, wherein the fiber has a diameter of 20 μm or less and a length of five times or more as much as the diameter.

13. The probe according to claim 11, wherein the fiber is at least one selected from the group consisting of carbon fiber, silicon carbide fiber, zinc oxide fiber and alumina fiber.

14. The probe according to claim 11, wherein the fiber is contained in the chloroprene-based resin in an amount of 20 to 70% by volume based on the total amount of the chloroprene-based resin and the fiber.

15. The probe according to claim 11, wherein the inorganic material is at least one selected from the group consisting of zinc oxide, zirconium oxide, aluminum oxide, silicon oxide, titanium oxide, silicon carbide, aluminum nitride, carbon and boron nitride.

16. The probe according to claim 1, wherein the acoustic absorbent layer has a thickness of 2 to 6 mm, a thermal conductivity at room temperature of 2 W/m·K or more, and an attenuation rate of 3 dB/mmMHz or more.

17. The probe according to claim 1, wherein t1/t2 which is a relation between the thickness t1 of the acoustic absorbent layer and the thickness t2 of the piezoelectric element is 8 to 15.

18. An ultrasonic diagnostic apparatus comprising a convex ultrasonic probe and an ultrasonic probe controller connected to the ultrasonic probe through a cable,
the ultrasonic probe comprising:
a backing member including a support which has a convexly curved surface and a thermal conductivity of 70 W/m·K or more, and an acoustic absorbent layer having a uniform overall thickness which is fixed on the convexly curved surface of the support;
a plurality of channels arranged on the acoustic absorbent layer of the backing member with spaces and each having a piezoelectric element and an acoustic matching layer formed on the piezoelectric element; and
an acoustic lens formed on the acoustic matching layer of each of the channels,
wherein a relation in which t1/t2 is 6 to 20 is satisfied, where t1 is the thickness of the acoustic absorbent layer and t2 is the thickness of the piezoelectric element.

19. The apparatus according to claim 18, wherein the acoustic absorbent layer of the ultrasonic probe includes an ethylene-vinyl acetate copolymer and at least one filler selected from the group consisting of carbon fiber which has a diameter of 20 μm or less and a length of five times or more as much as the diameter, silicon carbide fiber, zinc oxide fiber and alumina fiber contained in the ethylene-vinyl acetate copolymer, and the filler is contained in an amount of 20 to 70% by volume based on the total amount of the ethylene-vinyl acetate copolymer and the filler.

20. The apparatus according to claim 18, wherein the acoustic absorbent layer of the ultrasonic probe has a thickness of 2 to 6 mm, a thermal conductivity at room temperature of 2 W/m·K or more, and an attenuation rate of 3 dB/mmMHz or more.
